(19) United States
(12) Patent Application Publication
(10) Pub. No.: US 2022/0080414 A1
(43) Pub. Date: Mar. 17, 2022

(54) FLUID ANALYSIS WITH CHANNELS FORMED IN LIDS

(71) Applicant: Hewlett-Packard Development Company, L.P., Spring, TX (US)

(72) Inventors: Silam J. Choy, Corvallis, OR (US); Alexander Govyadinov, Corvallis, OR (US); Erik D. Torniainen, Corvallis, OR (US); Tod Woodford, Corvallis, OR (US)

(73) Assignee: Hewlett-Packard Development Company, L.P., Spring, TX (US)

(21) Appl. No.: 17/417,609

(22) PCT Filed: May 7, 2019
(86) PCT No.: PCT/US2019/031026

§ 371 Date: Jun. 23, 2021

(51) Int. Cl. B01L 3/00; G01N 21/64

(52) U.S. Cl. CPC B01L 3/502715; G01N 21/6428; B01L 2300/0883; B01L 2300/041; G01N 2201/062; B01L 2300/0645; B01L 2300/168; G01N 2021/6439; B01L 2300/0816

(57) ABSTRACT

In one example in accordance with the present disclosure, a fluid analysis device is described. The device includes a substrate, a die adhered to the substrate, and at least one fluid analysis element disposed on the die. A lid is adhered to the substrate and includes a channel formed thereinto be seated over the die. The device also includes an inlet port to the channel and an outlet from the channel. The inlet port and the outlet port are formed on at least one of the substrate and the lid. A number of electrical traces couple the die to a controller.

### BACKGROUND

Analytic chemistry is a field of chemistry that uses instruments to separate, identify, and quantify matter. In analytic chemistry, the fluid to be analyzed, or components therein are measured, chemically processed, and/or physically manipulated.

### BRIEF DESCRIPTION OF THE DRAWINGS

The accompanying drawings illustrate various examples of the principles described herein and are part of the specification. The illustrated examples are given merely for illustration, and do not limit the scope of the claims.

FIG. 1 is a block diagram of a fluid analysis device with a channel formed in a lid, according to an example of the principles described herein.

FIGS. 2A-2C are various views of a fluid analysis device with a channel formed in a lid, according to an example of the principles described herein.

FIGS. 3A and 3B are various views of a fluid analysis device with a channel formed in a lid, according to an example of the principles described herein.

FIG. 4 is a block diagram of a fluid analysis system with multiple fluid analysis devices with channels formed in lids, according to an example of the principles described herein.

FIG. 5 is a flow chart of a method for analyzing fluid in a lid-formed channel, according to an example of the principles described herein.

FIG. 6 is an isometric view of a fluid analysis device with a channel formed in a lid, according to another example of the principles described herein.

FIG. 7 is a top view of a fluid analysis device with a channel formed in a lid, according to another example of the principles described herein.

FIGS. 8A and 8B are various views of a fluid analysis device with a channel formed in a lid, according to another example of the principles described herein.

FIG. 9 is an isometric view of a fluid analysis device with a channel formed in a lid, according to another example of the principles described herein.

FIG. 10 is an isometric view of a fluid analysis device with a channel formed in a lid, according to another example of the principles described herein.

FIG. 11 is an isometric view of a fluid analysis device with a channel formed in a lid, according to another example of the principles described herein.

FIG. 12 is a cross-sectional view of a fluid analysis device with a channel formed in a lid, according to another example of the principles described herein.

FIG. 13 is a cross-sectional view of a fluid analysis device with a channel formed in a lid, according to another example of the principles described herein.

FIG. 14 is a cross-sectional view of a fluid analysis device with a channel formed in a lid, according to another example of the principles described herein.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

In analytic chemistry, fluid analysis elements are used to analyze fluids or components found within the fluids. For example, the components may be identified, measured, separated, or subject to a variety of fluid operations.

In some examples, fluid analysis devices such as sensors, actuators, or other components are used to analyze the fluid. According to the present specification, these processes can be performed in situ by a semiconductor die. As a specific example, measurements can be made by sensors disposed on the semiconductor die, chemical reactions can be initiated via a heater disposed on the semiconductor die, and physical manipulation of the fluid can be performed by micro-electro-mechanical systems (MEMS) components fabricated on the die.

In some examples, multiple fluid operations are carried out on a fluid. Accordingly, the present specification describes how a semiconductor die can be easily and effectively used, and inserted, into a microfluidic chamber as part of a larger fluid network.

Introducing die analysis functionality in a microfluidic application was performed by creating fluidic ports onto the die or to size the die to accommodate the size of the fluidic part. However, fluidic ports are generally larger (on the scale of greater than 0.5 mm) which constrains how small the die could be. Thus, many fluid analysis systems have a lower limit size based on the ports. In another example, such analysis dies are near, but not in direct contact with, the fluid being transported and manipulated in a fluid network. Accordingly, the results of any fluid analysis may be diminished, the results may include more error, and the overall capability of the die to act upon or measure the fluid is reduced.

Accordingly, the present specification describes a microfluidic channel with input/output flow ports. The microfluidic channel includes a die, such as a semiconductor die, that is in contact with the fluid passing between the ports. In one specific example, the die may be a complementary metal-oxide-semiconductor (CMOS) die mounted on a substrate. A lid is mounted over the die and onto the substrate with the ends thereof being sealed. Input and output ports may be created through the substrate or the lid and connected to the rest of the fluid network.

In this example, one end of the die extends out from the microfluidic chamber such that electrical signals and power connections can be provided to the die via electrical routing traces on the substrate.

In one particular example, the fluid analysis device is included in a microfluidic reaction chamber. A microfluidic reaction chamber refers to a chamber where a chemical reaction, or any other manipulation, processing, or sensing operation occurs. One such example of a reaction is the neutralization of a basic or acidic solution. Specifically, an input sample may have a certain pH and a user may want to change the pH based on downstream analysis. That is, the subsequent analysis operations may be most effective with solutions at a particular pH. Accordingly, a user may titrate various fluids together to change the pH of the solution. In another example, a subsequent operation may include an enzymatic reaction for which a particular pH is desired. Similarly, in this case, the pH of the enzyme may be changed in the sample preparation stage via the addition of another fluid.

The presence of a die in a microfluidic reaction chamber provides the ability to sense or measure properties of components of the fluid, or the fluid in the chamber. By inserting a semiconductor die into a microfluidic chamber, the semiconductor die is in direct contact with the fluid and can directly measure or act upon the fluid. In some examples, the die may be disposed in a long narrow microfluidic chamber. Accordingly, the die may also be long and narrow. Specifically, the die may be between 5 millimeters (mm) and 50 mm long while being between 50 micrometers (μM) and 1 mm wide.

As described above in some examples, multiple fluid operations are to be performed on a single fluid sample. These different fluid operations may be carried out by different sensors. In some examples, different sensors may be formed by processes or materials that are incompatible with one another such that it is not possible to place the fluid analysis elements on the same wafer. As a specific example, dichroic filters are placed over photodiodes for sensing components within a fluid. Different filters can be used to detect different components within a fluid. Having different filter properties for distinct photodiodes on the same die implements different thin film layers covering each photodiode. The processes to deposit and pattern these thin film layers at specific thicknesses and sizes over each photodiode on the same die may conflict among photodiodes and inhibit the performance of these thin films. Accordingly, the present specification by separating these photodiodes onto different wafers all while being within a single channel or reaction chamber avoids any associated manufacturing complexities and ensures enhanced performance of the sensors.

In another example, different types of sensors have different manufacturing processes. For example, an electrochemical sensor that includes electrodes may have different formation processes as compared to a photodiode with a dichroic filter. Similarly, the present specification includes both these components in a single reaction chamber albeit on different wafers.

In some examples, multiple parallel fluid chambers may be formed, each with a unique die/fluid analysis element. However, doing so splits the analyte which can result in an uneven concentration of analyte in the different chambers and/or a sufficient reduction in concentration and signal thus leading to imprecise analysis results.

Accordingly, the present specification splits the sensors onto multiple dies, which multiple sensors/dies may have different fabrication properties. In another example, by using a sliver die, such as the high aspect ratio sliver die described above, multiple fluid analysis devices may be placed on a single die due in part to the ability to sufficiently separate the components thereon. In either case, multiple die each with a different fluid analysis element, or a single sliver die with multiple fluid analysis elements disposed thereon, placing these dies themselves in the microfluidic channel or chamber, reduces the added cost of silicon from having multiple dies instead of just one and increases fluid analysis possibilities by allowing fluid analysis of incompatible sensors in a single reaction chamber.

Specifically, the present specification describes a fluid analysis device. The fluid analysis device includes a substrate, a die adhered to the substrate, and at least one fluid analysis element disposed on the die. The device also includes a lid adhered to the substrate. The lid has a channel formed therein to be seated over the die. The fluid analysis device also includes an inlet port to the channel and an outlet from the channel. The inlet port and the outlet port are formed on at least one of the substrate and the lid. The fluid analysis device also includes a number of electrical traces to couple the die to a controller.

The present specification also describes a fluid analysis system. The fluid analysis system includes multiple fluid analysis devices. Each fluid analysis device includes a substrate, a die adhered to the substrate, and at least one fluid analysis element disposed on the die. Each device also includes a lid adhered to the substrate, which lid has a channel formed therein to be seated over the die. Each fluid analysis device also includes an inlet port to the channel and an outlet from the channel. The inlet port and the outlet port are formed on at least one of the substrate and the lid. The fluid analysis device also includes a number of electrical traces to couple the die to a controller. In this example, each fluid analysis device is coupled to another fluid analysis device.

The present specification also describes a method. According to the method, fluid is received at an inlet of a channel, which inlet is formed in a lid disposed on top of a substrate. The fluid is passed through a channel formed in the lid over a die formed on the substrate. At least one fluid operation is performed on the fluid passing through the channel via at least one fluid analysis element disposed on the die. The fluid is then expelled through an outlet of the channel, which outlet is formed in the lid.

The systems and methods of the present specification 1) place a die/fluid analysis element in direct contact with the fluid to directly measure or act upon the fluid; 2) provide a long narrow die which increases die contact time with the fluid while the fluid flows through the channel, eliminate stagnant volume or air pockets in the channel when the channel is first filled with fluid, and increase surface area of the die in contact with the fluid for a given die footprint; 3) remove the size of the fluid connections as a constraint for fluid analysis systems; 4) place more than one planar surface of the die in contact with the fluid, thus increasing the performance of certain fluid analysis operations; 5) in some cases provide for multiple zones in the channel and on a single die to allow for distinct, and sequential fluid analysis operations; 6) allow sensors and other fluid analysis features that are otherwise incompatible to co-exist in the same microfluidic reaction chamber, by separating fluid analysis elements formed by conflicting fabrication processes onto separate dies; and 7) facilitate fabrication of dies of differing substrate material into a single package.

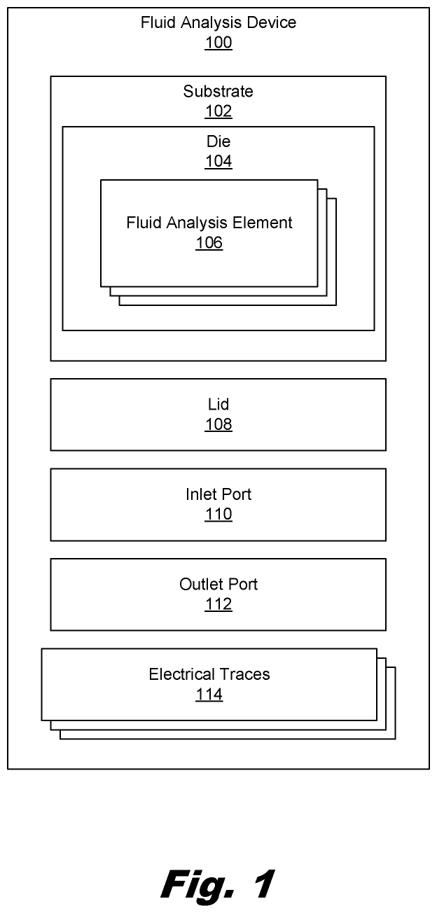

Turning now to the figures, FIG. 1 is a block diagram of a fluid analysis device (100) with a channel formed in a lid (108), according to an example of the principles described herein. In some examples, the fluid analysis device (100) is a microfluidic structure. In other words, the components, i.e., the die (104), fluid analysis elements (106), lid (108), inlet port (110), and outlet port (112) may be microfluidic structures. A microfluidic structure is a structure of sufficiently small size (e.g., of nanometer sized scale, micrometer sized scale, millimeter sized scale, etc.) to facilitate conveyance of small volumes of fluid (e.g., picoliter scale, nanoliter scale, microliter scale, milliliter scale, etc.).

The fluid analysis device (100) includes a substrate (102) on which other components of the fluid analysis device (100) are formed. The substrate (102) may be formed of a variety of materials including plastic, silicon, glass, metal, or any other rigid material such as a printed circuit board (PCB).

Disposed on top of the substrate (102) is a die (104), such as a semiconductor die (104). The die (104) provides a mounting surface for the fluid analysis elements (106) that operate on the fluid. The die (104) also provides power and data transmission paths between the fluid analysis elements (106) and the electrical traces (114). The electrical traces (114) couple the die (104) to a controller that provides the signals that control the fluid analysis elements (106). In some examples, the die (104) may be a high aspect ratio die (104). That is, the die (104) may be long and narrow. In some examples, the die (104) may have a length to width ratio of at least 3:1 and potentially greater such as 50:1. For example, the width of the die (104) may be 50 micrometers to 1 millimeter and the length of the die (104) may be from 5 millimeters to 50 millimeters. Using such a high aspect ratio die (104) allows for multiple fluid analysis elements (106) to be placed on the die (104) while allowing sufficient space between them to accommodate different fluidic operations.

In some examples, the fluid analysis elements (106) may be disposed serially along the die (104) such that sequential operations can be executed. For example, along a flow path a first analysis element (106) may be a heater to initiate a chemical reaction of components of the fluid. A second analysis element (106) along the flow path may be a sensor to analyze the fluid to determine a status, or result, of the chemical reaction. Given the length of the die (104), the fluid may be heated and enough time may pass as the fluid reaches the sensor for the initiated chemical reaction to occur. Moreover, by being narrow, and filling the channel, all of the fluid interacts with both components such that a complete reaction occurs. Thus, a long die (104) as described herein provides for linear fluidic operations to be performed on a single die (104).

As will be described below, in some examples the fluid analysis device (100) includes multiple die (104). These die (104) may be different types of die (104) that include fluid analysis elements (106) that are manufactured in different, and sometimes incompatible ways, or where the die (104) themselves are manufactured in different and potentially incompatible ways.

That is, the die (104) may include fluid analysis elements (106) that could not be formed on a traditional single die (104). However, due to the length of the high aspect ratio die (104) which adequately separates the elements, these fluid analysis elements (106) may be formed on a single die (104). That is, in some applications a period of time is desired between performing different operations. A high aspect ratio die (104) with multiple fluid analysis elements (106) disposed in sequential fashion along a flow path of the fluid provides a gap between fluid analysis elements (106), which gap allows for time-dependent sequential operations to be performed. All this may be done on a single die (104) in a single chamber rather than using multiple different fluid analysis devices (100) per operation.

At least one fluid analysis element (106) is disposed on a die (104) and in some examples multiple fluid analysis elements (106) may be disposed on a die (104). In the case of one fluid analysis element (106) per die (104), the fluid analysis device (100) may include multiple die (104) such that multiple fluid analysis operations can be executed. In the case of multiple fluid analysis elements (106) per die (104), the fluid analysis device (100) may include one or multiple die (104).

The fluid analysis elements (106) may be of a variety of types and may therefore carry out a variety of operations. Some examples include lysing elements that rupture cell walls, heaters that raise the temperature of fluid, sensing elements that detect the presence of certain fluids, or certain components within a fluid, electrochemical sensors, optical elements, physical manipulators that could mix, cool, separate, filter or interrupt the flow path of the fluid. Another example of a fluid analysis element (106) is a chemical agent applied to the surface of the die (104) or to a pad mounted on the die (104). This chemical agent may react with biochemical reagents in a fluid that capture different proteins. Different kinds of chemical agents may be added to perform any number of chemical analysis/manipulation operations.

While particular reference is made to a few specific types of fluid analysis elements (106), any type, and any number of fluid analysis elements (106) may be disposed on the die (104), whether the single or multiple fluid analysis elements (106) are disposed on a single or multiple die (104).

The fluid analysis device (100) also includes a lid (108) that is adhered to the substrate (102). Formed in the lid (108) is a channel. That is, during fabrication a recess is formed in the lid (108). This channel is seated over the die (104). In this way, fluid that passes through the channel is passed over the die (104), thus exposing the fluid to the fluid analysis elements (106) disposed thereon such that the fluid may be acted upon. The lid (108) and the substrate (102) may form a microfluidic reaction chamber to hold a volume of at least one fluid. In such a chamber any number of reactions may be effectuated, such as the aforementioned lysing, physical manipulation, chemical alteration, sensing, etc. The fluid analysis device includes an inlet port (110) to introduce fluid into the channel and an outlet port (112) to expel fluid from the channel. The inlet port (110) and outlet port (112) may be formed on at least one of the substrate (102) and the lid (108).

Thus, the present fluid analysis device (100) allows for direct contact of a fluid with the fluid analysis elements (106) on a die (104) and does so without being constrained by the size of large fluidic connections.

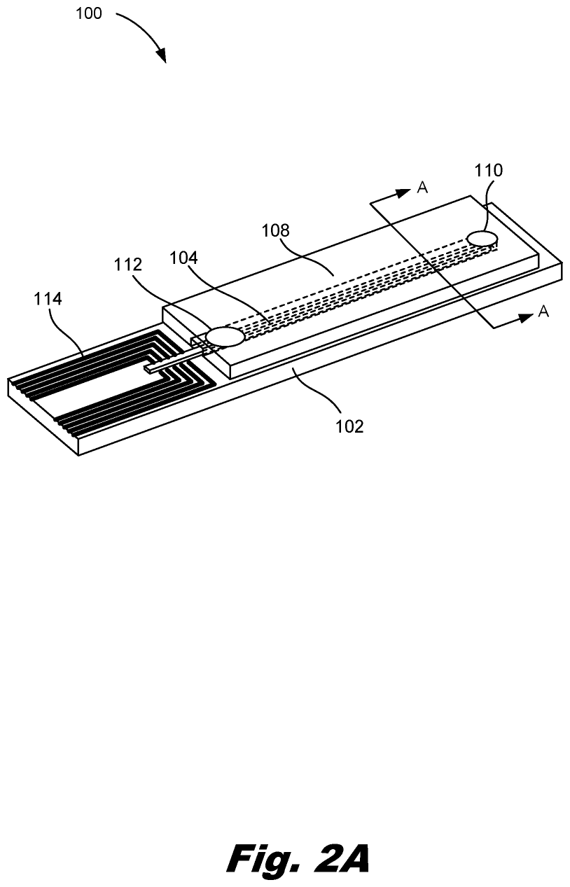

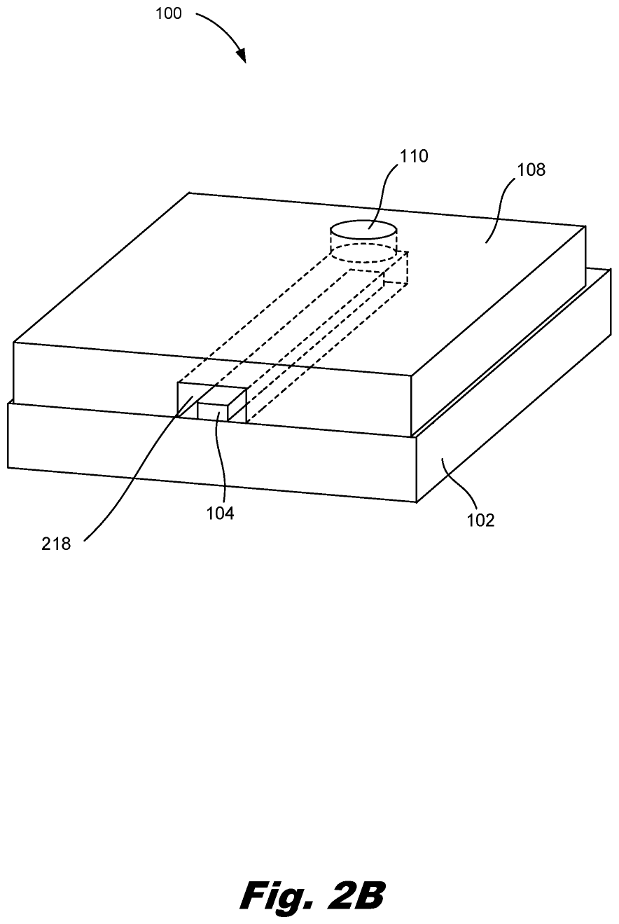

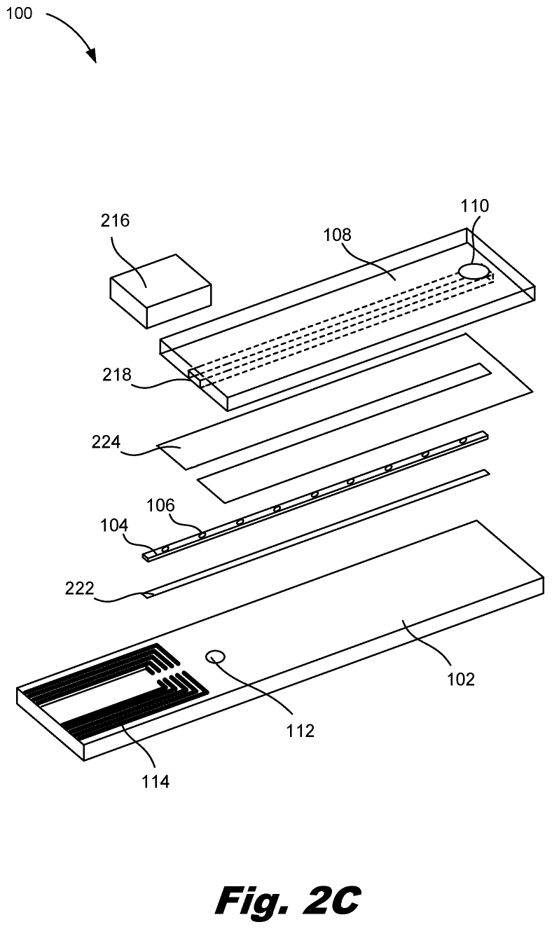

FIGS. 2A-2C are various views of a fluid analysis device (100) with a channel (218) formed in a lid (108), according to an example of the principles described herein. Specifically, FIG. 2A is an isometric view, FIG. 2B is a cross-sectional view taken along the line A-A from FIG. 2A, and FIG. 2C is an exploded view. FIG. 2A clearly depicts the substrate (102) and die (104). Note that as depicted in FIG. 2A, the substrate (102) and the die (104) extend outside of the lid (108) such that electrical connections can be formed with the portion of the die (104) disposed within the lid (108) being depicted in dashed lines.

FIG. 2A clearly depicts the high aspect ratio layout of the die (104) which is much longer than it is wide. That is, the die (104) may be at least 10 times longer than it is wide. FIG. 2A also depicts the inlet (110) and outlet (112) from which fluid is transported to/from other fluid analysis devices of an overall fluid analysis system. In the example depicted in FIG. 2A, the inlet (110) and outlet (112) are formed in the lid (108). However, in other examples, either of these components may be formed in the substrate (102).

FIG. 2A also depicts the electrical traces (114) which route signals and power to the die (104). These electrical traces (114) can be formed in a variety of ways including laser defined structure operations and as molded lead frames. While specific reference is made to a few particular methods of trace formation, a variety of other methods may be implemented. The electrical traces (114) may be formed on different surfaces. In the example depicted in FIG. 2A, the electrical traces (114) are formed on the substrate (102), but in other examples, the electrical traces (114) may be formed on the lid (108).

The lid (108) of the fluid analysis device (100) may be formed of a variety of materials. Depending on the application, in some examples the lid (108) may be an optically transparent lid (108). In this example optical signals and/or light may pass through the optically transparent lid (108) to illuminate the fluid passing therethrough, or to aid in any of the fluid analytic/manipulation operations that are executed. Examples of optically transparent materials include glass and polycarbonate.

FIG. 2B is a cross-sectional view which clearly indicates the die (104) disposed within a channel (218). As described above, the channel (218) may be a microfluidic structure. For example, the channel (218) may contain less than 10 microliters of fluid at any point in time. As a specific example, the die (104) may have cross-sectional dimensions of 200 micrometers by 200 micrometers. In this example, the channel (218) may have a cross-sectional area of 600 micrometers by 400 micrometers. That is, a spacing between the channel (218) walls and the die (104) may be at least as great as a dimension of the die (104).

As described above, in some examples, the lid (108) may be formed of an optically transparent material. In other examples, the lid (108) may be formed of another material such as SUB. In this example, the channel (218) may be fabricated during the manufacturing operation for the die (104).

As described above, the inlet (110) and outlet (112) may be formed in a variety of places. In the example depicted in FIG. 2C, the inlet (110) is disposed on the lid (108) and the outlet (112) is disposed on the substrate (102). That is the outlet ports (112) are disposed on one of the substrate (102) or the lid (108) and the inlet ports (110) are disposed on the other.

FIG. 2C also depicts a die adhesive (222) that is used to bond the die (104) to the substrate (102). FIG. 2C also depicts a lid adhesive (224) that is used to bond the lid (108) to the substrate (102).

FIG. 2C also depicts multiple fluid analysis elements (106) disposed on the die (104). For simplicity in demonstration, a single instance of a fluid analysis element (106) is indicated with a reference number. In this example, fluid is introduced into the channel (218) through the inlet (110). As the fluid travels towards an outlet (112), multiple fluid operations are performed on the fluid. In the example depicted in FIGS. 2A-2C where just one die (104) is disposed within the channel (218), this includes passing the fluid by multiple fluid analysis elements (106), each of which are disposed on a single die (104). FIG. 2C also depicts the lid (108) with the channel (218) on the underside indicated in dashed lines.

In some examples, the fluid analysis device (100) also includes an encapsulant (216) disposed over the electrical connection between the die (104) and the electrical traces (114). The connection between the die (104) and the electrical traces (114) may be wire-bonded and in some cases can be fragile. The encapsulant (216) protects the mechanical and electrical robustness of this interface. The encapsulant (216) also serves to seal on end of the channel (218).

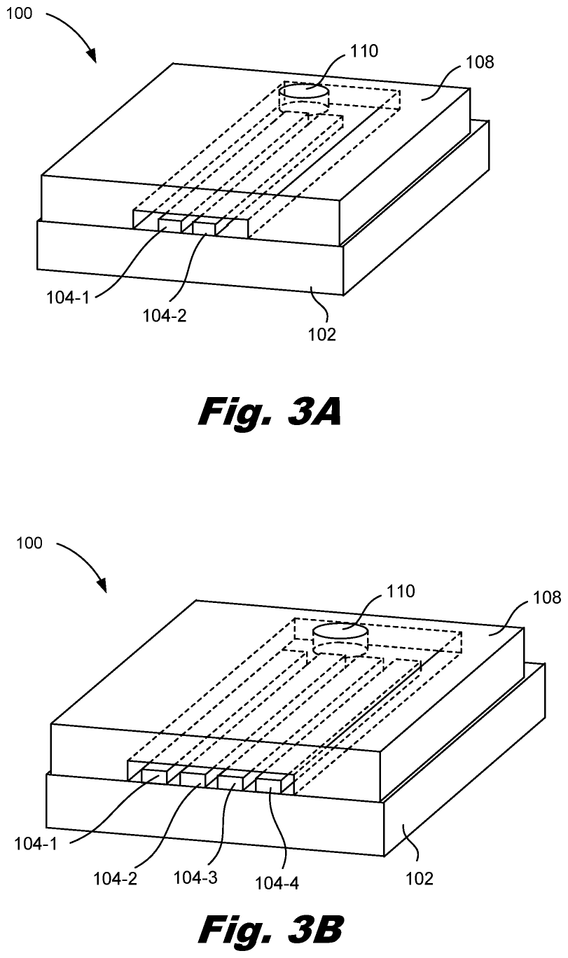

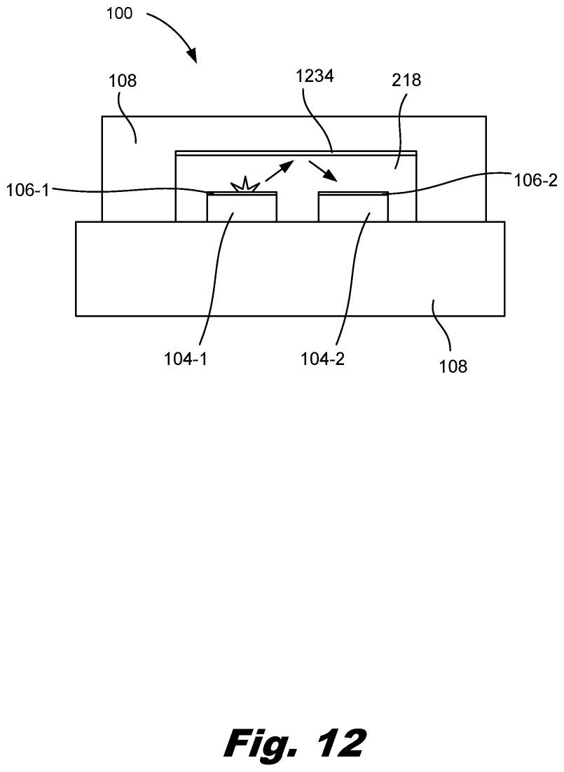

FIGS. 3A and 3B are various views of a fluid analysis device (100) with a channel (FIG. 2B, 218) formed in a lid (108), according to an example of the principles described herein. Specifically, FIGS. 3A and 3B depict examples of a fluid analysis device (100) that include multiple die (104), two in FIG. 3A and four in FIG. 3B, formed on the substrate (102) in the channel (FIG. 2A, 218). That is, FIG. 3A depicts an example where two die (104-1, 104-2) are seated under a channel (FIG. 2A, 218) in the lid (FIG. 1, 108) and FIG. 3B depicts an example where four die (104-1, 104-2, 104-3, 104-4) are seated under a channel (FIG. 2A, 218) in the lid (FIG. 1, 108). In this example, each die (104) may be processed from different wafers having a different set of fabrication processes. In this example, each die (104) is at least one of an independent physical structure or includes a distinct fluid analysis element (FIG. 1, 106) disposed thereon. Examples of these arrangements are provided below in connections with FIGS. 12-14.

The channel (FIG. 2A, 218) may be sized differently depending on the number of die (104) to be enclosed therein. In the example depicted in FIG. 3A with two die (104-1, 104-2) and the example depicted in FIG. 3B with four die (104-1, 104-2, 104-3, 104-4), the die may be separated by a distance equal to the width of each die (104). For example, for die (104) having a width of 200 micrometers, the die (104) may be separated by a gap of at least 400 micrometers. Accordingly, a width of the channel (FIG. 2A, 218) depicted in FIG. 3A may be 1200 micrometers and a width of the channel (FIG. 2A, 218) depicted in FIG. 3B may be 2400 micrometers. Notes that these values are examples and a variety of channel (FIG. 2A, 218) dimensions may be implemented in accordance with the principles described herein.

During use, fluid is introduced into the channel (FIG. 2A, 218) through the inlet (110). As it travels towards an outlet (FIG. 1, 112), multiple fluid operations are performed on the fluid. In the example depicted in FIGS. 3A and 3B where each die (104) is has a distinct fluid analysis element (FIG. 1, 106), this includes passing the fluid by multiple fluid analysis elements (106), each of which are disposed on a different die (104).

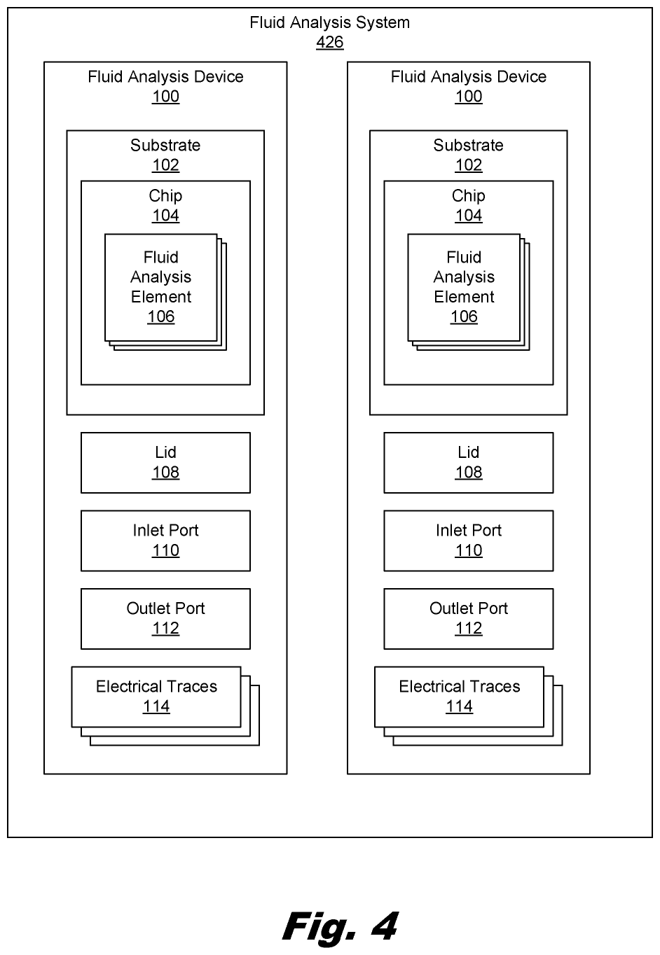

FIG. 4 is a block diagram of a fluid analysis system (426) with multiple fluid analysis devices (100) with channels (FIG. 2A, 218) formed in lids (108), according to an example of the principles described herein. That is, a fluid analysis system (426) may be made up of multiple components, at least some of which are fluid analysis devices (100). The fluid analysis devices (100) may be modular and combinable in any number of fashions to generate a fluid analysis network. Such a fluid analysis system (426) is highly customizable. For example, different fluid analysis devices (100) may perform certain operations based on the fluid analysis elements (106) disposed thereon. These different fluid analysis devices (100) may be directly or indirectly coupled to one another or to other components of the system (426). That is, a fluid analysis device (100) may be coupled to another fluid analysis device (100). In some examples, one of the fluid analysis devices (100) may be coupled to a different component such as a pump, a vent port, a fluid inlet or a waste receptable. That is, the present specification describes a fluid analysis system (426) wherein different fluid analysis devices (100) can be combined in any number of fashions to carry out any complex sequence of fluid analysis operations desired for a particular application.

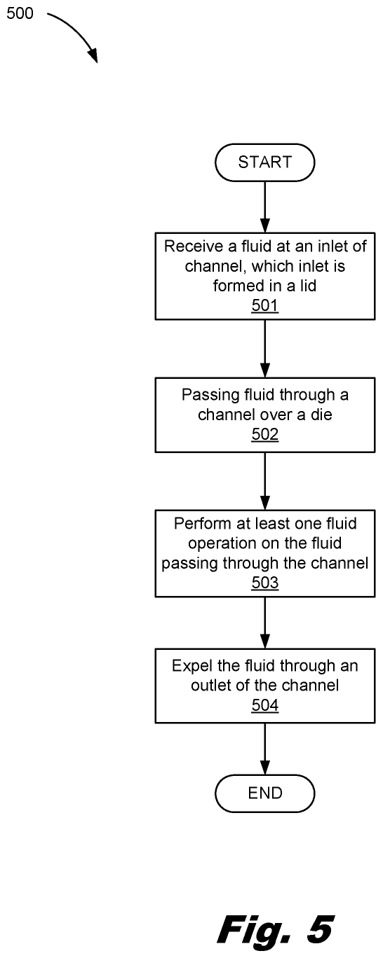

FIG. 5 is a flow chart of a method (500) for analyzing fluid in a lid-formed channel (FIG. 2A, 218), according to an example of the principles described herein. According to the method (500), fluid is received (block 501) at an inlet (FIG. 1, 110) of a channel (FIG. 2A, 218). As described above, in some examples, the inlet (FIG. 1, 110) is formed in a lid (FIG. 1, 108) disposed on top of a substrate (FIG. 1, 102). The fluid is then passed (block 502) through a channel (FIG. 2A, 218) that is formed in the lid (FIG. 1, 108) over a die (FIG. 1, 104) formed on the substrate (FIG. 1, 102). In so doing, at least one fluid operation is performed (block 503) on the fluid passing through the channel (FIG. 2A, 218) by at least one fluid analysis element (FIG. 1, 106) that is disposed on the die (FIG. 1, 104).

That is, as described above, the die (FIG. 1, 104) may be long and narrow such that multiple fluid analysis elements (FIG. 1, 106) may be placed in line along a flow path of the fluid. Accordingly, multiple fluid operations can sequentially be performed on a particular fluid sample. For example, as the fluid passes through the channel (FIG. 2A, 218) it may pass by a first fluid analysis element (FIG. 1, 106) which may be a heater to initiate a chemical reaction. As the fluid continues to flow, it may sequentially pass by a second fluid analysis element (FIG. 1, 106) which may be a mechanical element to mix the fluid components that underwent the chemical reaction. A third fluid analysis element (FIG. 1, 106) may be a sensing component that determines the presence of the output of the chemical reaction. In this example, once fluid has been acted upon it is expelled (block 504) through the outlet (FIG. 1, 112) of the channel (FIG. 2A, 218) where it can be subsequently operated on. That is the outlet (FIG. 1, 112) which may be on the lid (FIG. 1, 108), substrate (FIG. 1, 102), or other component, may be coupled to another analysis device of the fluid analysis system. Thus, the method (500) may be repeated for multiple modules in a customized fluid analysis system (FIG. 4, 426)

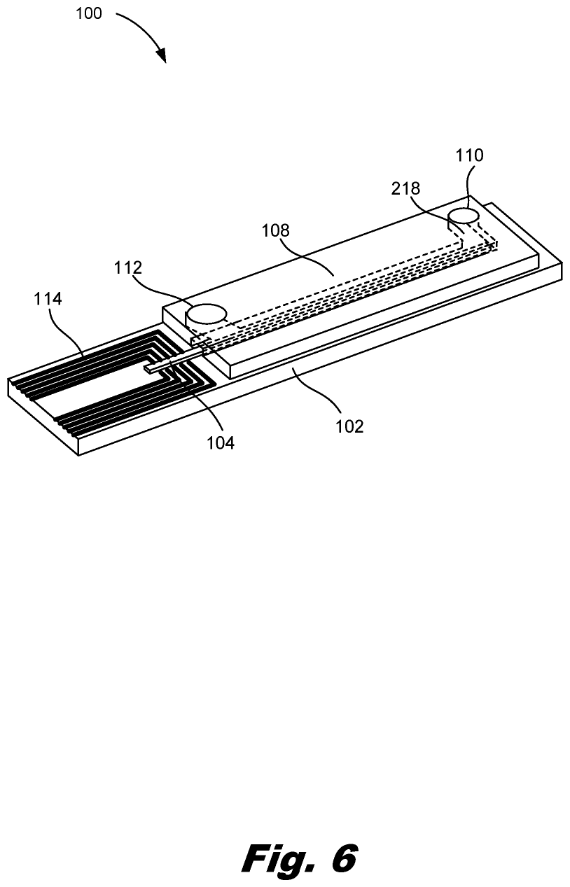

FIG. 6 is an isometric view of a fluid analysis device (100) with a channel (218) formed in a lid (108), according to another example of the principles described herein. FIG. 6 depicts components previously described such as the electrical traces (114), outlet (112), lid (108), substrate (102), and inlet (110). FIG. 6 also depicts the die (104) on which the fluid analysis elements (FIG. 1, 106) are disposed.

In the example depicted in FIG. 6 however, the channel (218) is a non-straight channel (218) and the inlet (110) and outlet (112) may not be disposed over the die (104). Doing so may increase the accuracy of any fluid analysis performed as well as preserve the longevity of the fluid analysis device (100). That is, fluid is driven through the system using any number of mechanisms. As the fluid passes through an inlet (110) it may crash into the underlying substrate (102). Such a crashing force may damage the surface which it contacts. By not placing the inlet (110) directly over the die (104), the fluid crashes into the substrate (102) and not into the potentially more fragile die (104) thus preventing wear on the die (104) and/or fluid analysis elements (FIG. 1, 106) as well as avoiding any potential mechanical failure of the die (104), each of which could affect the functionality of the fluid analysis device (100).

Such an offset inlet (110) may also alter the flow dynamics of the fluid. That is, fluid entering the inlet (110) may have a certain velocity that is undesired for the fluid analysis operation of the fluid analysis device (100). Accordingly, the offset inlet (110) may slow the flow into the channel (218) and in some cases may affect its characteristics, i.e., amount of turbulent flow, to a desired state before entering the channel (218) for fluid analysis.

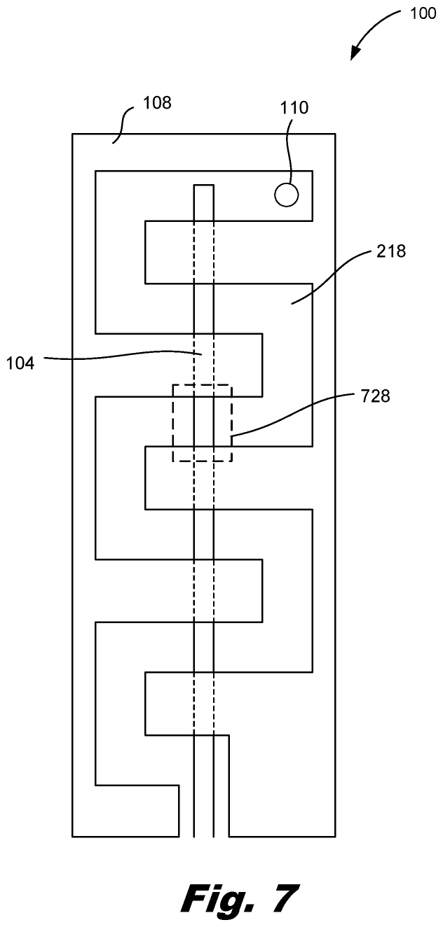

FIG. 7 is a top view of a fluid analysis device (100) with a channel (218) formed in a lid (108), according to another example of the principles described herein. In the example depicted in FIG. 7, the channel (218) is a serpentine channel (218) which crosses the die (104). In FIG. 7, the portions of the die (104) indicated in dashed lines are those parts that are under the lid and not exposed in the channel (218). In this example, zones (728) are defined on the die (104), a zone (728) being defined as a region of the die (104) that is separated spatially and temporarily from another zone (728). As described above, the fluid analysis device (100) may include multiple fluid analysis elements (FIG. 1, 106) disposed on the die (104). In the example depicted in FIG. 7, each fluid analysis element (FIG. 1, 106) may be disposed within a zone (728). For simplicity in illustration, just one zone (728) is indicated with a reference number. Being that different zones (728) may have different fluid analysis elements (FIG. 1, 106) which may carry out different fluid analysis operations, different actions and processes can be performed in each separated die (104) zone (728). The serpentine channel (218) may also create a greater spatial offset between fluid operations than a channel (218) that is straight and directly over the die (104).

During use, fluid is introduced into the channel (218) through the inlet (110). As it travels towards an outlet (FIG. 1, 112), multiple fluid operations are performed on the fluid. In the example depicted in FIG. 7 where each die (104) has multiple zones (728) with fluid analysis elements (FIG. 1, 106), this includes passing the fluid by multiple zones (728) of the die (104) which die (104) is formed on the substrate (FIG. 1, 102). The definition of zones (728) via a serpentine channel (218) over a die (104) provides for a more effective fluid analysis device (100). That is, rather than having multiple die (104) aligned in series each to perform a different operation, one die (104) may have multiple zones (728) each performing a different operation.

In some examples, additional adhesive is placed over the die (104) and under the lid (108) in regions that separate the serpentine turns, for example over the dashed regions of the die (104) in FIG. 7. Doing so creates a barrier between adjacent zones (728) except through the channel (218). That is, this adhesive seals each segment of the serpentine channel (218) from each other.

In some examples, the lid (108) may take a variety of topographical characteristics. For example, the lid (108) may be thinner in the areas not disposed over the die (104). That is, the lid (108) may be thinner in the serpentine bends. In one particular example, this may allow for a more rapid thermal dissipation such that the fluid in the serpentine bends may cool more rapidly.

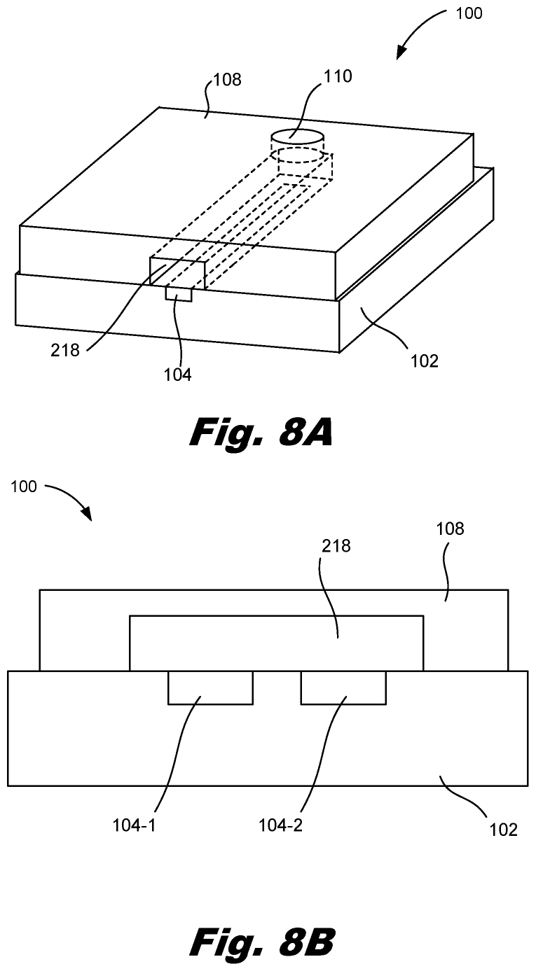

FIGS. 8A and 8B are various views of a fluid analysis device (100) with a channel (218) formed in a lid (108), according to another example of the principles described herein. Specifically, FIG. 8A is an isometric cross-sectional view of a fluid analysis device (100) with one die (104) and FIG. 8B is a front cross-sectional view of a fluid analysis device (100) with two die (104-1, 104-2). In some examples, as depicted in FIGS. 8A and 8B, the die (104) are embedded into the substrate (102). Doing so provides a planar surface for sealing over the die (104). Accordingly, the channel (218) height may be lower, thus accommodating even smaller fluidic structures as the height of the lid (108) may be reduced due to embedding the die (104) in the substrate (102). The embedding of the die (104) in the substrate (102) may also simplify manufacturing. For example, as described in FIG. 7, additional adhesive may be used in a serpentine channel (218) to isolate serpentine segments. However, if the die (104) is embedded as depicted in FIGS. 8A and 8B, the sealing surfaces on which this adhesive is disposed is flat and therefore much more accommodating to the sealing of distinct zones (FIG. 7 728).

To form embedded die (104), the die (104) may be overmolded during a fabrication of the substrate (102). For example, the substrate (102) may be an epoxy mold compound which is in a liquid form prior to curing. When in a liquid form, the epoxy mold compound may be formed around the die (104) and then cured to enclose the die (104) in the substrate. This operation may also avoid the use of the adhesive to affix the die (104) to the substrate (102).

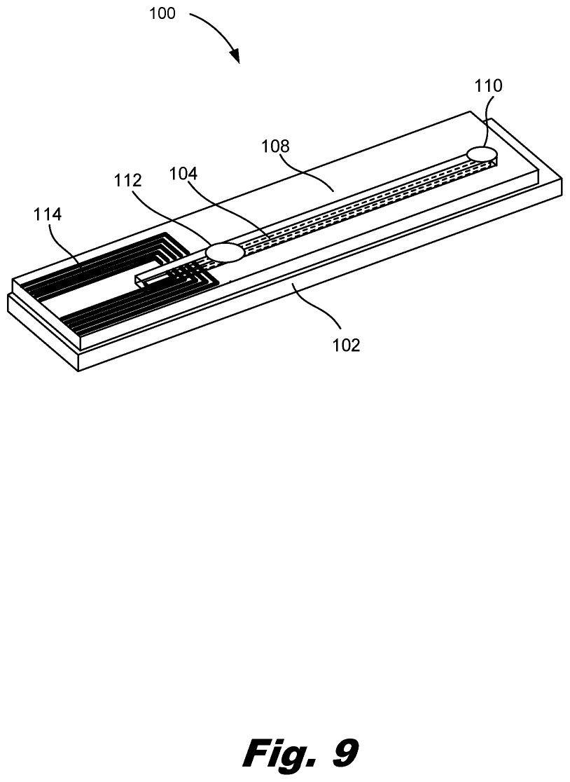

FIG. 9 is an isometric view of a fluid analysis device (100) with a channel (FIG. 2A, 218) formed in a lid (108), according to another example of the principles described herein. In the example depicted in FIG. 9, the electrical traces (114) are formed in the lid (108). That is, as described above, the electrical traces (114) may be formed in either the lid (108) or the substrate (102). Doing so provides for greater flexibility in fluid analysis device (100) usage. For example, placing electrical traces (114) in the lid (108) allows for the substrate (102) to be metallic, which lid (108) may be a glass or other insulating material.

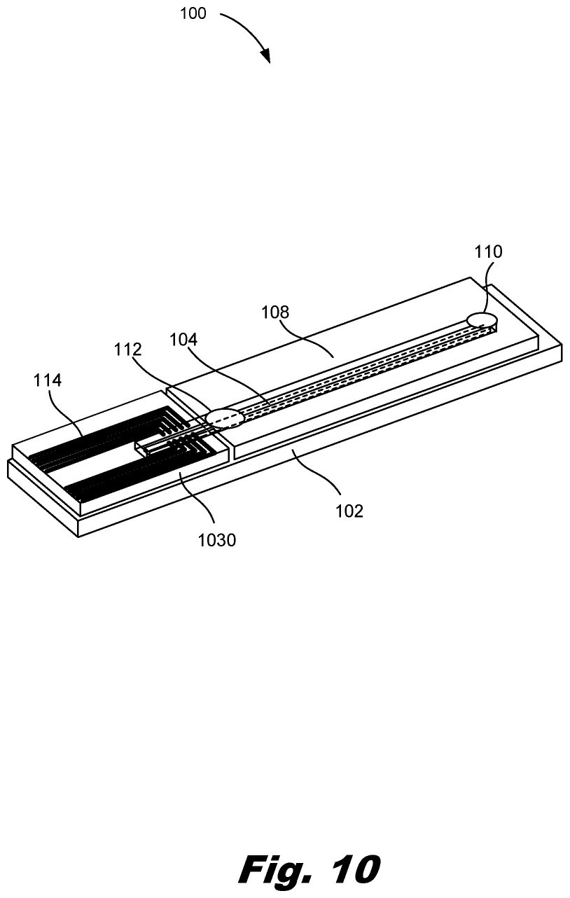

FIG. 10 is an isometric view of a fluid analysis device (100) with a channel (FIG. 2A, 218) formed in a lid (108), according to another example of the principles described herein. In the example depicted in FIG. 10, the electrical traces (114) are formed on a separate substrate. That is, each fluid analysis device (100) includes a second substrate which is adhered to the substrate (102). The electrical traces (114) are formed on this second substrate. In the example depicted in FIG. 10, the second substrate is a printed circuit board (1030).

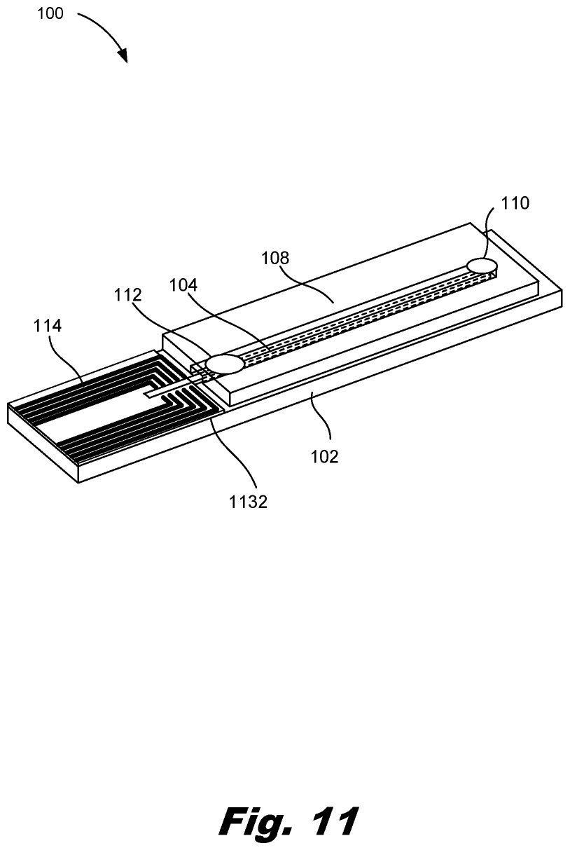

FIG. 11 is an isometric view of a fluid analysis device (100) with a channel (FIG. 2A, 218) formed in a lid (108), according to another example of the principles described herein. Similar to the example depicted in FIG. 10, in this example, the electrical traces (114) are formed on a separate substrate. In this example, the second substrate is a flexible circuit (1132). As described above, in some examples the electrical traces (114) are wire bonded to the die (104). In other examples, the electrical traces (114) may be bonded in other ways such as tape-automated bonding or a thermocompression bond between cantilevered leads on the flexible circuit (1132) to pads on the die (104).

FIG. 12 is a cross-sectional view of a fluid analysis device (100) with a channel (218) formed in a lid (108), according to another example of the principles described herein. Specifically, as described above in some examples the die (104) may be formed of different materials and may have different fluid analysis elements (106) disposed thereon. The fluid analysis elements (106) may be elements that would be incompatible to put on the same die (104) either due to processing incompatibility or to mounting surface incompatibility. For example, a first die (104-1) may be a die of one material such as gallium arsenide, indium gallium nitride, gallium phosphide, and aluminum gallium arsenide that includes a light-emitting diode (LED) analysis element (106-1) disposed thereon and the second die (104-2) may be a silicon die with a photodiode analysis element (106-2) disposed thereon. In this example, light emitted from the LED analysis element (106-1) may reflect off a reflective coating (1234) in the channel (218) and collected at the photodiode analysis element (106-2) to form a detection system.

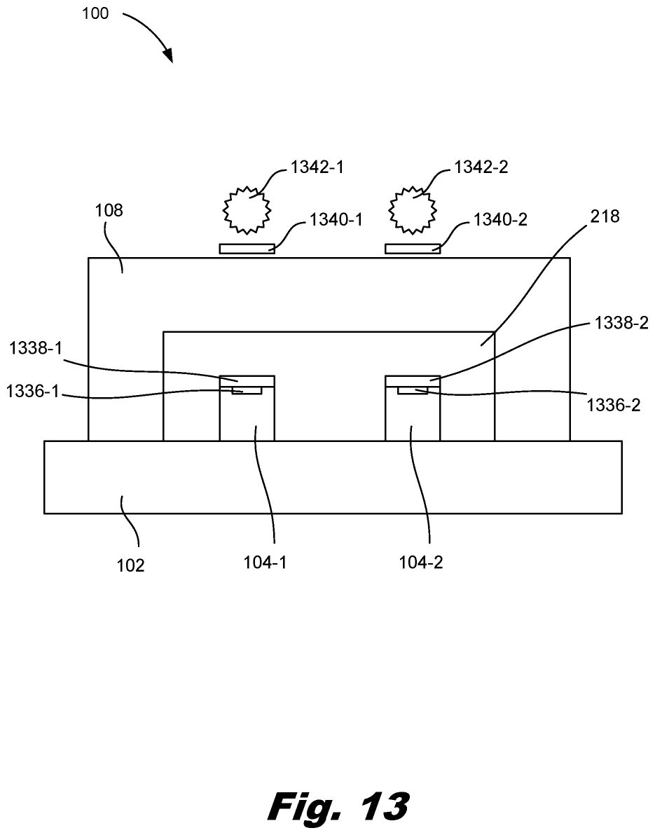

FIG. 13 is a cross-sectional view of a fluid analysis device (100) with a channel (218) formed in a lid (108), according to another example of the principles described herein. As described above, the fluid analysis device (100) may be used for a variety of purposes. One such purpose is to optically detect the presence and/or concentration of certain compounds in a fluid passing therethrough. In these examples, components within a fluid sample are tagged with fluorescent or color markers. Once in the channel (218), these markers can be detected by shining light into the channel (218), through the fluid onto photodiodes (1336). Accordingly, a system includes light sources (1342), filters (1340), and dichroic filters (1338) and photodiodes (1336) disposed on different die (104-1, 104-2).

In some examples, different types of these components can detect different color or fluorescent markers. For example, as depicted in FIG. 13, a first light source (1342-1) may be a blue illumination light-emitting diode (LED) that is directly above a green dichroic filter (1338-1) disposed over a first photodiode (1336-1) and a second light source (1342-2) may be a green illumination LED that is directly above a red dichroic filter (1338-2) disposed over a second diode (1336-2). Thus, the fluid analysis system can detect different fluorescent markers to detect different compounds within a fluid sample. In other fluid analysis systems, such dual-detection would be performed in two separate fluid analysis devices (100). However, using the fluid analysis device (100) with multiple die (104-1, 104-2) and associated mounted fluid analysis elements (FIG. 1, 106), multiple sensing operations can be carried out in a single reaction chamber.

A specific example of the operation of the fluid analysis device (100) depicted in FIG. 13 is now presented. Specifically, an example of the operation of one detection system is described. In this example, light emitted by a first light source (1342-1) may be centered around a particular wavelength such as 470 nanometers, but may be a broad curve. Light passes through a first dichroic filter (1340-1) to a narrower spectrum. Fluorophore in a fluid has an excitation wavelength and emission which overlap and may emit light at a different wavelength, for example, 520 nanometers. The first filter (1338-1) blocks light of a certain wavelength, for example below 500 nanometers, so that just light emitted from the fluorophore reaches the diode (1336-1). That is, the light from the first light source (1342-1) may be much higher intensity than the light emitted from the fluorophore. Accordingly, if filters (1338) are not used, the photodiode (1336) would be blinded by the source (1342) and the light from the fluorophore would not be detectable. The second detection system (-2) operates in a similar fashion, albeit at different wavelengths.

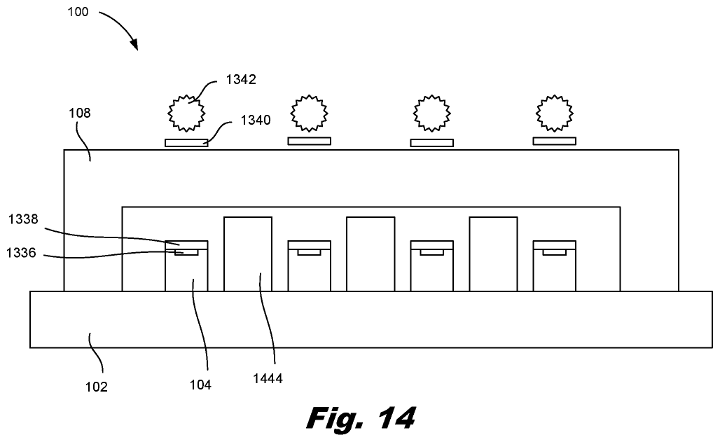

FIG. 14 is a cross-sectional view of a fluid analysis device (100) with a channel (218) formed in a lid (108), according to another example of the principles described herein. FIG. 14 is similar to FIG. 13, except that FIG. 14 depicts more detection systems. For simplicity, a single instance of the light source (1342), filter (1340), dichroic filter (1338), and photodiode (1336) are depicted with reference numbers. In this example, a violet light source (1342) and filter (1340) are directly above a blue dichroic filter (1338) over a photodiode (1336); a blue light source (1342) and filter (1340) are directly above a green dichroic filter (1338) over a photodiode (1336); a green light source (1342) and filter (1340) are directly above a red dichroic filter (1338) over a photodiode (1336); and a red light source (1342) and filter (1340) are directly above a violet dichroic filter (1338) over a photodiode (1336). Thus, using the fluid analysis device (100) with multiple die (104) and associated mounted fluid analysis elements (FIG. 1, 106), multiple sensing operations can be carried out in a single reaction chamber.

In some examples, as depicted in FIG. 14, the fluid analysis device (100) includes a partition (1444) disposed between adjacent die (104). The partition (1444) thermally isolates the adjacent die (104). That is, without such partitions (1444) there may be thermal crosstalk throughout the channel (218) which may affect fluid analysis of the different fluid analysis elements (FIG. 1, 106).

In some examples, the partitions (1444) may be formed of the same material as the substrate (102) and in other examples may be a different material. In either case, the partition (1444) material may be a low thermally conductive material to prevent the transfer of heat energy to adjacent die (104). Moreover, while FIG. 14 depicts a particular partition (1444) height, the partitions (1444) may be of any height.

The systems and methods of the present specification 1) place a die/fluid analysis element in direct contact with the fluid to directly measure or act upon the fluid; 2) provide a long narrow die which increases die contact time with the fluid while the fluid flows through the channel, eliminate stagnant volume or air pockets in the channel when the channel is first filled with fluid, and increase surface area of the die in contact with the fluid for a given die footprint; 3) remove the size of the fluid connections as a constraint for fluid analysis systems; 4) place more than one planar surface of the die in contact with the fluid, thus increasing the performance of certain fluid analysis operations; 5) in some cases provide for multiple zones in the channel and on a single die to allow for distinct, and sequential fluid analysis operations; 6) allow sensors and other fluid analysis features that are otherwise incompatible to co-exist in the same microfluidic reaction chamber, by separating fluid analysis elements formed by conflicting fabrication processes onto separate dies; and 7) facilitate fabrication of dies of differing substrate material into a single package.

What is claimed is:

1. A fluid analysis device, comprising:
a substrate;
a die adhered to the substrate;
at least one fluid analysis element disposed on the die;
a lid adhered to the substrate, the lid having a channel formed therein to be seated over the die;
an inlet port to the channel;
an outlet from the channel, wherein the inlet port and the outlet port are formed on at least one of the substrate and the lid; and
a number of electrical traces to couple the die to a controller.

2. The fluid analysis device of claim 1, wherein the channel is a serpentine channel which crosses the die to define zones on the die.

3. The fluid analysis device of claim 1, further comprising multiple fluid analysis elements disposed on the die, each fluid analysis element disposed within a zone.

4. The fluid analysis device of claim 1, wherein the die is embedded into the substrate.

5. The fluid analysis device of claim 1, wherein the outlet ports are disposed on the substrate or lid and inlet ports are disposed on the other.

6. The fluid analysis device of claim 1:
wherein the lid and substrate form a microfluidic reaction chamber to hold a volume of at least one fluid; and
the fluid analysis device comprises multiple dies formed on the substrate in the channel.

7. The fluid analysis device of claim 6, wherein each die is at least one of:
an independent physical structure; and
comprises a distinct fluid analysis element disposed thereon.

8. The fluid analysis device of claim 6, further comprising a partition disposed between adjacent dies.

9. A fluid analysis system comprising:
multiple fluid analysis devices, each fluid analysis device comprising:
a substrate;
a die adhered to the substrate;
at least one fluid analysis element disposed on the die;
a lid adhered to the substrate, the lid having a channel formed therein to be seated over the die;
at least one inlet port to receive fluid into the channel;
at least one outlet port to expel fluid from the channel, wherein the at least one inlet port and the at least one outlet port are formed on at least one of the substrate and the lid; and
a number of electrical traces extending outside of the lid to couple the die to a controller,
wherein at least one fluid analysis device is coupled to another fluid analysis device.

10. The fluid analysis system of claim 9, wherein the electrical traces are formed in at least one of the lid and the substrate.

11. The fluid analysis system of claim 9, wherein:
each fluid analysis device further comprises a second substrate adhered to the substrate; and
the electrical traces are formed on the second substrate.

12. The fluid analysis system of claim 9, wherein the lid of at least one fluid analysis device is an optically transparent lid.

13. A method, comprising:
receiving a fluid at an inlet of a channel, wherein the inlet is formed in a lid disposed on top of a substrate;
passing the fluid through a channel formed in the lid over a die formed on the substrate;
performing at least one fluidic operation on the fluid passing through the channel via at least one fluid analysis element disposed on the die; and
expelling the fluid through an outlet of the channel, wherein the outlet is formed in the lid.

14. The method of claim 13, further comprising passing the fluid by multiple zones of the die formed on the substrate.

15. The method of claim 13, further comprising performing multiple fluidic operations by performing at least one of:
passing the fluid by multiple fluid analysis elements, each disposed on a single die; and
passing the fluid by multiple fluid analysis components, each disposed on a different die.
